(19) United States
(12) Patent Application Publication
(10) Pub. No.: US 2004/0086463 A1
(43) Pub. Date: May 6, 2004

(54) Nasal solution containing a broad spectrum microbicide and a method for its use

(76) Inventor: Richard L. Hansen, Mentor, OH (US)

Correspondence Address: Laura F. Shunk, Hudak, Shunk & Farine Co. LPA, Suite 307, 2020 Front Street, Cuyahoga Falls, OH, 44221, US

(21) Appl. No.: 10/690,242

(22) Filed: Oct. 21, 2003

Related U.S. Application Data
(63) Continuation-in-part of application No. 10/139,522, filed on May 6, 2002, now Pat. No. 6,664,289.

(51) Int. Cl. A61L 9/04; A61K 31/315; A61K 33/14

(52) U.S. Cl. 424/45; 424/661; 514/494

(57) ABSTRACT

The invention provides for a nasal spray which is an aqueous solution containing a broad spectrum microbicide which may be applied in nasal passages in the manner of a decongestant type nose spray in the event of known or suspected exposure of the individual to common cold virus, flu, or other infective microbial agents. The solution contains active agents which are based on a chlorine or hypochlorite solution, or a solution of chlorine or hypochlorite plus a salt of chloride, bromide or iodide. Alternatively, the solution may contain a bromine solution, or a solution of bromine plus a salt of chloride, bromide or iodide such as sodium chloride, zinc chloride, sodium bromide, zinc bromide, sodium iodide or zinc iodide. The chlorine, hypochlorite, bromine, chloride, bromide, and iodide may originally come from inorganic compounds or organic compounds, which are then dissolved in the water. Further, the solution may contain glycerin or another moisturizing or wetting agent for the nasal mucosa. Zinc gluconate or a zinc halide such as zinc chloride, zinc bromide or zinc iodide may also be included.

### FIELD OF THE INVENTION

The invention relates generally to an aqueous nasal solution for use in treatment of and also the prevention of infection by microbes (like a head cold, flu, or other infection) and generally includes a broad-spectrum microbicide specifically including aqueous chlorine or bromine, hypochlorite ion and/or chloride, bromide or iodide ion. In a further embodiment, aqueous sodium hypochlorite can be used by itself or with the addition of glycerin or glycol for the treatment and prevention of infection.

### SUMMARY OF THE INVENTION

The invention relates generally to aqueous solution which contains a broad spectrum microbicide which may be applied in nasal passages in the manner of a decongestant type nose spray. The spray is intended particularly for human use. It is to be applied in the event of known or suspected exposure of the individual to common cold virus, flu, or other infective microbial agents including for example, bacteria, rickettsia, viruses and even mold and fungus. It may also be applied when there is a pre-existing infection caused by the previously mentioned agents. The solution contains an active agent which is based on a chlorine or hypochlorite solution, or a solution of chlorine or hypochlorite plus a salt of chloride, bromide or iodide. Alternatively, the solution may contain a bromine solution, or a solution of bromine plus a salt of chloride, bromide or iodide such as sodium chloride, zinc chloride, sodium bromide, zinc bromide, sodium iodide or zinc iodide. The chlorine, hypochlorite, bromine, chloride, bromide, and iodide may originally come from inorganic compounds or organic compounds, which are then dissolved in the water. Further, the solution may contain glycerin or another moisturizing or wetting agent for the nasal mucosa since the halogens or halides may be dehydrating or drying. Zinc gluconate or a zinc halide such as zinc chloride, zinc bromide or zinc iodide may also be included to further render the nasal mucosa more slippery and thus more difficult for invading microbes to colonize the area.

### BACKGROUND OF THE INVENTION

Cold viruses or other microbes are sometimes transmitted by aerosol such as by a water droplet that is dispersed by coughing or sneezing, or by personal contact including, for example, hand to hand contact where a handshake transfers the microbes from one person to another and then the contaminated hand is brought into the vicinity of the individual's nose. (As when touching the nose or scratching it or rubbing it.) It is generally believed that infective agents will grow and multiply in regions where they have the proper temperature, nutrients, and other conditions conducive to growth or multiplication such as in the nasal mucosa. Thus, an object of the present invention is to apply a broad-spectrum microbicide to the area where infective viruses may be transmitted or transported and would ordinarily multiply.

A number of explanations could be proposed for the effectiveness of the microbicide in reducing the risk of full-blown infection. For example, the composition may act to kill the microbe, or to render it ineffective. Further, the microbicide may act to slow diffusion into a cell by tagging it with a heavy ion. This slowing of diffusion may slow the microbe's multiplication and allow the body's natural defenses to catch up and eliminate the microbes. Further, the nasal solution may dilute the attacking organism and wash it out away from the optimum area for growth so as to weaken the statistical probability of success of the infection.

It is thus an object of the invention to apply a medical agent to the area of entry, namely the nose, and specifically the inside of the nose, the nasal fossae and the sinus areas within the nose. This treatment can be remedial so as to inhibit an existing infection, or prophylactic so as to prevent spread into the respiratory system by affecting the microbe in the nose. Thus, in accordance with a method of using the nasal spray, it could be applied as soon as the person has determined he or she has “caught” a cold. It could also be applied after the person first begins to feel the initial symptoms of catching a cold, such as stuffiness or muscular aches or fever. Or it could be applied prophylactically as soon as a person has been exposed to others who have colds and has real reason to be concerned about catching a cold after such an exposure. Moreover, it could be applied in a similar manner for cases of influenza or other infections for minimizing symptoms and extent of “infection”.

### DETAILED DESCRIPTION OF THE INVENTION

One preferred embodiment of the invention is an aqueous solution of sodium hypochlorite with a halogen salt such as sodium chloride, sodium bromide, sodium iodide, zinc chloride, zinc bromide or zinc iodide. It may also include more than one of the aforementioned salts. In addition, it may also include zinc gluconate.

Another embodiment of the invention would be an aqueous solution of chlorine with a halogen salt such as sodium chloride, sodium bromide, sodium iodide, zinc chloride, zinc bromide or zinc iodide. It may also include more than one of the aforementioned salts. In addition, it may also include zinc gluconate.

Another preferred embodiment of the invention is an aqueous solution of bromine with a halogen salt, i.e. chloride, bromide or iodide. The salt may constitute a chloride, a bromide or iodide with a specific example being sodium bromide, or zinc bromide.

Another embodiment of this invention is the use of organic compounds of chlorine, bromine or iodine, or organic compounds that are chlorides, bromides or iodides. These compounds are such that they decompose in solution or chemically react in order to provide the necessary halogen components for making the aqueous mixture that will be used to spray into the nose onto the nasal mucosa.

There may be particular advantages of using mixed halogens since they sometimes combine in stable but reactive trimers like Br₂Cl—, Br₂I—, I₂Cl—, I₂Br—, etc. Having stabilized the reactive halogen, there will likely be more of the halogen present in the solution for reaction with sensitive areas of the microbes existing on the nasal mucosa.

It is postulated that these ionic species will react chemically with organic or biological chemicals on or in the virus so as to kill it or render it ineffective. Further, the solutions can contain glycerin or some other additive to act as a moisturizer since the halogen species tend to have a dehydrating, drying effect on the nasal mucosa. The pH could be adjusted, if necessary, to match the pH of mucous membranes or to optimize the activity without being harmful to the nasal mucosa. It can be adjusted up or down using sodium hydroxide, chlorine, sodium hypochlorite, hydrogen chloride, bromine, or hydrogen bromide. Thus, an appropriate pH for the final solution would be from about pH 5.5 to about pH 8.5, and preferably from about pH 6 to pH 8 and more preferably from about pH 6.5 to pH 7.5.

Sodium hypochlorite would be present in a concentration of about 0.5 ppmw to about 50,000 ppmw, and preferably from about 5 ppmw to about 5000 ppmw and more preferably from about 50 ppmw to 500 ppmw. It may also be potassium, calcium, or other hypochlorites.

Sodium or potassium chloride would be present in a concentration of about 55 ppmw to 75,000 ppmw, and preferably from 250 ppmw to 50,000 ppmw and more preferably from about 2500 ppmw to 25,000 ppmw. This could also be magnesium or calcium chloride or other chlorides.

Sodium or potassium bromide would be present in a concentration of about 0.7 ppmw to about 140,000 ppmw, more preferably from about 140 ppmw to about 55,000 ppmw, and more preferably from about 1,400 ppmw to about 28,000 ppmw. This could also be magnesium or calcium bromide or other bromides.

Sodium or potassium iodide would be present in a concentration of about 0.1 ppmw to about 160,000 ppmw, more preferably from about 200 ppmw to about 80,000 ppmw, and more preferably from about 2,000 ppmw to about 40,000 ppmw. This could also be magnesium or calcium iodide or other iodides.

Chlorine would be present at a concentration of about 0.4 ppmw to about 50,000 ppmw, more preferably from about 2 ppmw to about 5000 ppmw, and more preferably from about 10 ppmw to about 500 ppmw.

Bromine would be present at a concentration of about 1 ppmw to about 75,000 ppmw, more preferably from about 4 ppmw to about 8,000 ppmw, and more preferably from about 20 ppmw to about 1000 ppmw.

Zinc chloride would be present at a concentration of about 0.4 ppmw to about 50,000 ppmw, more preferably from about 40 ppmw to about 15,000 ppmw, and more preferably from about 400 ppmw to about 5,000 ppmw.

Zinc bromide would be present at a concentration of about 0.7 ppmw to about 80,000 ppmw, more preferably from about 75 ppmw to about 33,000 ppmw, and more preferably from about 750 ppmw to about 10,000 ppmw.

Zinc iodide would be present at a concentration of about 1 ppmw to about 120,000 ppmw, more preferably from about 100 ppmw to about 80,000 ppmw, and more preferably from about 1,000 ppmw to about 20,000 ppmw.

Zinc gluconate would be present at a concentration of about 1.7 ppmw to about 60,000 ppmw, more preferably from about 17 ppmw to about 6,000 ppmw, and more preferably from about 170 ppmw to about 1800 ppmw.

Glycerine would be present at a concentration of about 0.01 wt % to about 5 wt %, more preferably from about 0.04 wt % to about 2 wt %, and more preferably from about 0.1 wt % to about 1 wt %. Propylene glycol could be used as a substitute for glycerine.

### EXAMPLES:

### Example 1

An example solution was mixed up and used by a 52 year-old male for the alleviation of head cold symptoms, including the typical nasal congestion. The applications were started early enough in the progress of the cold development that subsequent cold symptoms were relatively minor. It is not known to what extent the treatments altered the total course of the cold development, including its symptoms and extent. In order to provide a more concrete description of the example, the following represents a list in detail of the manner in which the solution was made.

First of all, normal bleach, which is a NaOCl (sodium hypochlorite) solution of strength 5.25%, was obtained. The bleach was chosen to avoid some of the new versions that are marketed that include “lemon freshening”, etc. in order to avoid potential side effects from those scenting chemicals. A small quantity of the bleach was diluted to ⅕th of its original strength by adding 8 drops of the bleach to 32 drops of water to make a total solution of 40 drops of which 8 represent the original bleach. A separate container was filled with 45 milliliters of water. 3 drops of the 1.05% bleach solution were added to the water. The solution was thoroughly mixed and put into a nasal spray bottle.

The spray was administered by spraying 2 to 4 times briskly into the nose while inhaling slightly. This was repeated approximately every 4 hours as needed. The subject observed a reduction of the mucosa production, as well as a lessening of the irritation of the back of the nose and pharynx, including the tonsils and adenoids. The nose and sinuses continued to decongest with further application of the spray and other cold symptoms were reduced or alleviated.

### Example 2

An example solution was mixed up and used by a 52 year-old male for the alleviation of head cold symptoms, including the typical nasal congestion. The applications were started after a couple of days into the progress of the cold. It is not known to what extent the treatments altered the total course of the cold development, including its symptoms and extent. In order to provide a more concrete description of the example, the following represents a list in detail of the manner in which the solution was made.

First of all, normal bleach, which is a NaOCl (sodium hypochlorite) solution of strength 5.25%, was obtained. The bleach-was chosen to avoid some of the new versions that are marketed that include “lemon freshening”, etc. in order to avoid potential side effects from those scenting chemicals. A small quantity of the bleach was diluted to ⅕th of its original strength by adding 8 drops of the bleach to 32 drops of water to make a total solution of 40 drops of which 8 represent the original bleach. 45 milliliters of water was placed into a separate container. Then 3 drops of the 1.05% bleach solution was added to the 45 milliliters of water. The solution was thoroughly mixed. Finally, 3 drops of glycerine were added to the solution. The solution was thoroughly mixed and put into a nasal spray bottle.

The spray was administered by spraying 2 to 4 times briskly into the nose while inhaling slightly. This was repeated approximately every 4 to 5 hours as needed. The subject observed a noticeable reduction of the mucosa production, as well as a lessening of the irritation of the back of the nose and pharynx. The nose and sinuses continued to decongest with further application of the spray and other cold symptoms were reduced or alleviated.

### Example 3

An example solution could be mixed up for use by a 53 year-old male for the alleviation of head cold symptoms, including the typical nasal congestion. The applications would be started after about 2 days into the progress of the cold. In order to provide a more concrete description of the example, the following represents a list in detail of the manner in which the solution would be made.

First of all, normal bleach, which is a NaOCl (sodium hypochlorite) solution of strength 5.25%, would be obtained. The bleach would be chosen to avoid some of the new versions that are marketed that include “lemon freshening”, etc. in order to avoid potential side effects from those scenting chemicals. A small quantity of the bleach would be diluted to ⅕th of its original strength by adding 8 drops of the bleach to 32 drops of water to make a total solution of 40 drops of which 8 represent the original bleach. A separate solution would be made up by dissolving 0.5 gram of NaBr (sodium bromide) crystals in 45 milliliters of water. Once the solute was thoroughly dissolved, then 3 drops of the 1.05% bleach solution would be added. The solution would be thoroughly mixed. Finally, 3 drops of glycerine would be added to the solution. The solution would be thoroughly mixed and put into a nasal spray bottle.

The spray would be administered by spraying 2 to 4 times briskly into the nose while inhaling slightly. This would be repeated approximately every 4 to 5 hours as needed. It is expected that the subject would observe a noticeable reduction of the mucosa production, as well as a lessening of the irritation of the back of the nose and pharynx. It is also expected that the nose and sinuses would continue to decongest with further application of the spray and other cold symptoms would be reduced or alleviated.

### Example 4

An example solution could be mixed up for use by a 53 year-old male for the alleviation of head cold symptoms, including the typical nasal congestion. The applications would be started after about 2 days into the progress of the cold. In order to provide a more concrete description of the example, the following represents a list in detail of the manner in which the solution would be made.

First of all, liquid bromine, 99.5+% which is at present available from Aldrich Chemicals would be used to prepare a ˜3% w/w aqueous solution, by mixing 1 drop of bromine with 99 drops of water. (Note that bromine has an approximate density of 3.11 g/ml at 20 degrees Centigrade.) A separate solution would be made up by dissolving 1 gram of NaCl (sodium chloride) crystals in 45 milliliters of water. Once the solute was thoroughly dissolved, then 1 drop of the ˜3% bromine solution would be added. The solution would be thoroughly mixed. Finally, 3 drops of glycerine would be added to the solution. The solution would be thoroughly mixed and put into a nasal spray bottle.

The spray would be administered by spraying 2 to 4 times briskly into the nose while inhaling slightly. This would be repeated approximately every 4 to 5 hours as needed. It is expected that the subject would observe a noticeable reduction of the mucosa production, as well as a lessening of the irritation of the back of the nose and pharynx. It is also expected that the nose and sinuses would continue to decongest with further application of the spray and other cold symptoms would be reduced or alleviated.

While in accordance with the patent statutes the best mode and preferred embodiment have been set forth, the scope of the invention is not limited thereto, but rather by the scope of the attached claims.

What is claimed is:

1. A method of reducing the risk of microbial infection or of combating a microbial infection which is established first in the nasal mucosa area comprising:
the application of a nasal spray solution within the nose, said nasal spray solution comprising an aqueous solution having following components:
a) sodium hypochlorite in a concentration of about 0.5 ppmw to about 50,000 ppmw; and
optionally one or more of zinc gluconate at a concentration of about 1.7 ppmw to about 60,000 ppmw, and glycerine or propylene glycol at a concentration of about 0.01 wt % to about 5 wt %.

2. A method of reducing the risk of microbial infection or of combating a microbial infection which is established first in the nasal mucosa area comprising:
the application of a nasal spray solution within the nose, said nasal spray solution comprising an aqueous solution having following components:
a) chlorine in a concentration of about 0.4 ppmw to about 50,000 pp mw; and
optionally one or more of zinc gluconate at a concentration of about 1.7 ppmw to about. 60,000 ppmw, and glycerine or propylene glycol at a concentration of about 0.01 wt % to about 5 wt %.

3. A method of reducing the risk of microbial infection or of combating a microbial infection which is established first in the nasal mucosa area comprising:
the application of a nasal spray solution within the nose, said nasal spray solution comprising an aqueous solution having following components:
a) bromine in a concentration of about 1 ppmw to about 75,000 ppmw; and
optionally one or more of zinc gluconate at a concentration of about 1.7 ppmw to about 60,000 ppmw, and glycerine or propylene glycol at a concentration of about 0.01 wt % to about 5 wt %.

4. A method of reducing the risk of microbial infection or of combating a microbial infection which is established first in the nasal mucosa area comprising:
the application of a nasal spray solution within the nose, said nasal spray solution comprising an aqueous solution having following components:
a) sodium hypochlorite in a concentration of about 0.5 ppmw to about 50,000 ppmw;
b) from about 0.1 ppmw to 160,000 ppmw of a salt selected from one or more of sodium chloride and sodium bromide; and
optionally one or more of zinc gluconate at a concentration of about 1.7 ppmw to about 60,000 ppmw, and glycerine or propylene glycol at a concentration of about 0.01 wt % to about 5 wt %.

5. A method of reducing the risk of microbial infection and also of combating an established microbial infection which is established first in the nasal mucosa area comprising the application of a nasal spray solution within the nose, said nasal spray solution comprising an aqueous solution having;
a) from about 0.4 ppmw to about 50,000 ppmw of chlorine; and
b) from about 0.7 ppmw to 140,000 ppmw of a salt selected from one or more of sodium chloride or sodium bromide; and
optionally one or more of zinc gluconate at a concentration of about 1.7 ppmw to about 60,000 ppmw, and glycerine or propylene glycol at a concentration of about 0.01 wt % to about 5 wt %.

6. A method of reducing the risk of microbial infection and of combating a microbial infection which is established first in the nasal mucosa area comprising the application of a nasal spray solution within the nose, said nasal spray solution comprising an aqueous solution having one or more of the following components:
a) from about 1 ppmw to about 75,000 ppmw of bromine; and
b) from about 0.7 ppmw to 140,000 ppmw of a salt selected from one or more of sodium chloride or sodium bromide; and
optionally one or more of zinc gluconate at a concentration of about 1.7 ppmw to about 60,000 ppmw, and glycerine or propylene glycol at a concentration of about 0.01 wt % to about 5 wt %.

7. A nasal spray solution comprising an aqueous solution having following components:
a) bromine in a concentration of about 1 ppmw to about 75,000 ppmw; and
optionally one or more of zinc gluconate at a concentration of about 1.7 ppmw to about 60,000 ppmw, and glycerine or propylene glycol at a concentration of about 0.01 wt % to about 5 wt %.

8. The nasal spray solution as set forth in claim 7 wherein the bromine is present in a concentration of about 40 to about 8,000 ppmw.

9. The nasal spray solution as set forth in claim 8 wherein the bromine is present in a concentration of about 20 to about 1,000.

10. A nasal spray solution comprising an aqueous solution having following components:
a) sodium hypochlorite in a concentration of about 0.5 ppmw to about 50,000 ppmw;
b) from about 0.1 ppmw to 160,000 ppmw of a salt selected from one or more of sodium chloride and sodium bromide; and
optionally one or more of zinc gluconate at a concentration of about 1.7 ppmw to about 60,000 ppmw, and glycerine or propylene glycol at a concentration of about 0.01 wt % to about 5 wt %.

11. The nasal spray solution as set forth in claim 10 wherein component a) is present in a concentration of from about 5 to about 5,000 ppmw, and component b) is present in a concentration of about 140 to about 55,000 ppmw.

12. The nasal spray solution as set forth in claim 11 wherein component a) is present in a concentration of from about 50 to about 500 ppmw, and component b) is present in a concentration of about 1,400 to about 28,000 ppmw.

13. A nasal spray solution comprising an aqueous solution having the following components;
a) from about 0.4 ppmw to about 50,000 ppmw of chlorine; and
b) from about 0.7 ppmw to 140,000 ppmw of a salt selected from one or more of sodium chloride or sodium bromide; and
optionally one or more of zinc gluconate at a concentration of about 1.7 ppmw to about 60,000 ppmw, and glycerine or propylene glycol at a concentration of about 0.01 wt % to about 5 wt %.

14. The nasal spray solution as set forth in claim 13 wherein component a) is present in a concentration of from about 2 to about 5,000 ppmw, and component b) is present in a concentration of about 140 to about 55,000 ppmw.

15. The nasal spray solution as set forth in claim 14 wherein component a) is present in a concentration of from about 10 to about 500 ppmw, and component b) is present in a concentration of about 1,400 to about 28,000 ppmw.

16. A nasal spray solution comprising an aqueous solution having the following components:
a) from about 1 ppmw to about 75,000 ppmw of bromine; and
b) from about 0.7 ppmw to 140,000 ppmw of a salt selected from one or more of sodium chloride or sodium bromide; and
optionally one or more of zinc gluconate at a concentration of about 1.7 ppmw to about 60,000 ppmw, and glycerine or propylene glycol at a concentration of about 0.01 wt % to about 5 wt %.

17. The nasal spray solution as set forth in claim 16 wherein component a) is present in a concentration of from about 40 to about 8,000 ppmw, and component b) is present in a concentration of about 140 to about 55,000 ppmw.

18. The nasal spray solution as set forth in claim 17 wherein component a) is present in a concentration of from about 20 to about 1,000 ppmw, and component b) is present in a concentration of about 1,400 to about 28,000 ppmw.
